(19) United States
(12) Patent Application Publication
(10) Pub. No.: US 2003/0181891 A1
(43) Pub. Date: Sep. 25, 2003

(54) Magnetic instrument bar

(76) Inventors: Cheryl A. Cooper, Fort Smith, AR (US); Robyn K. Stafford, Fort Smith, AR (US)

Correspondence Address: CHERYL COOPER, RR 1 BOX 1139, ROLAND, OK, 74954, US

(21) Appl. No.: 10/106,295

(22) Filed: Mar. 25, 2002

(51) Int. Cl. A61B 17/00

(52) U.S. Cl. 606/1

(57) ABSTRACT

The magnetic instrument bar comprises a rounded top elongated horizontal bar with a flat bottom and ends consisting of three layers of material, the outer most layer is a durable rubber material, the middle layer is a durable magnetic material, with the inner most layer is a durable stainless steel material. All materials used have the integrity to withstand repeated sterilization. Thus, creating a smooth rounded flat bottom magnetic instrument holder. It safely maintains instruments in an upright position allowing easy identification and accessability for passing instruments during all procedures.

### FIELD OF THE INVENTION

The present invention broadly relates instrument holder devices and techniques for surgical and non-surgical instruments. Specifically, the present invention is a Magnetic Instrument Bar used to organize and support instruments (e.g., medical instruments) in a upright position on a flat support surface such as a table in an operating room.

### BACKGROUND—DESCRIPTION OF PRIOR ART

A variety of instruments are used in the operating room. Such instruments are often placed on a table or Mayo stand propped up with a rolled towel. Sometimes the instruments extend over the lip of the Mayo stand, and sometimes the instruments are simply laid flat on the stand. The types of instruments handled in this manner are varied and include, for example, hemostats, scissors, scalpels, forceps, etc.

This procedure, however, has several disadvantages. For example, the instruments are not supported in an upright manner. As a result, they often fall over and are therefore more difficult to identify and to grasp. Further, when the instruments are simply laying on the table they occupy more space than is desired.

Also, after instruments are used and are placed back on the table, there is no time to line up and try to support the instruments in an upright manner. Thus, when it is necessary to use any of such instruments again, it is necessary to locate the proper instrument on the table and try to grasp it. This can be difficult, cumbersome, and time-consuming. Also, after an instrument has once been used in the operation it will have blood or other body fluids on it. This can make the instrument more difficult to grasp when it is needed again in the operation.

The prior system of laying the instruments on a Mayo stand or table is cumbersome and inefficient. The instruments overlap each other and to fall over on their side. Consequently, the proper instruments are difficult to locate and grasp when needed. Although the use of a rolled cloth towel is helpful for propping up one end of the instruments on a stand or table, most hospitals no longer use cloth towels because of the expense and for laundering. Paper towels do not work for supporting instruments in an elevated manner because they do not have sufficient internal strength to support the weight of the instruments.

There has not heretofore been provided a convenient and effective magnetic holder for supporting instruments on the back table or Mayo stand which enables such instruments to readily and easily grasped when needed.

A patentability search was conducted on this device and the most pertinent patens uncovered were as follows:

Artzer U.S. Pat No. 5,201,430, Duffy U.S. Pat. No. 3,557,791, Loveland U.S. Pat. No. 3,889,670, Ackering U.S. Pat. No. 4,003,378, Evans U.S. Pat. No. 4,018,217, Mayer U.S. Pat. No. 4,275,719, Salyer U.S. Pat. No. 5,236,433, Lackman U.S. Pat. No. 5,582,607, Lechot U.S. Pat. No. 568,290.

All above patents disclose instrument or device holders, some of which could be employed to hold instruments and others which obviously could not. The use of magnetic's to hold various instruments is well known in the art; however the present device is an improvement of the state of the art as will be apparent from the detailed description which follows. Conventionally, a rolled surgical towel is used to support instruments on both the back table and mayo stand. Use of the conventionally rolled surgical towel method is problematic due to weight of the surgical instruments, the towel collapses and instruments unvariably fall from an upright position to their side making it difficult and cumbersome to pick out a particular instrument. Thus, creating delays in passing instruments to the surgeon.

The prior art fails to disclose a single purpose magnetic instrument bar for a mayo stand and or back table solely for supporting instruments during surgical procedures.

### SUMMARY OF THE INVENTION

My invention is an improved and simple to use magnetic instrument bar for holding instruments safely upright via magnetic force during surgical procedures. The present comprises a elongated horizontal bar which has a flat bottom, rounded top, and is magnetic. It is light weight, cost effective, and simple to use.

To use my magnetic instrument bar the back table and Mayo stand are draped out in the usual fashion with sterile coverings, the magnetic instrument bar is then positioned on the back table and or Mayo stand then instruments (e.g., hemostats of all sizes and styles, scissors, forceps, clamps, needle holders, etc.) placed on the magnetic instrument bar allowing the instruments to remain upright. The magnetic instrument bar can be provided in various sizes and styles, as desired. Instruments can then be adjusted and removed safely and easily as needed.

The instruments can be easily and readily placed onto the magnetic instrument bar without having to locate or position an instrument in a specific area. Rather, the instrument will will automatically hold on its own when it placed against the bar.

The device operates in the manner of a instrument holder wherein the surgical instruments will be held upright via magnetic force. Therefore, a primary object of the present invention is to provide a instrument holder that will hold instruments in place for surgical and non-surgical procedures.

(a) Another primary object of the present invention is to provide a magnetic instrument bar which simplifies successful and safe transference of instruments during surgical and non-surgical procedures.

(b) A related object of the present invention is to provide a rounded, magnetic instrument bar for holding instruments which is small and easy to use for all surgical and non-surgical procedures.

(c) an object of the present invention is to provide a magnetic instrument bar which will maintain itself in position without being held.

(d) An object of the present invention is to provide a magnetic instrument bar which will eliminate the need for a conventionally rolled surgical towel, which sole task is to hold instruments.

(e) A related object of the present invention is to provide a magnetic instrument bar which will reduce the costs associated with all surgical procedures.

(f) Specifically, an object of the present invention is to provide a magnetic instrument bar which will reduce cost by reducing the number of towels used and instruments that are dropped off the field due to towels collapsing under weight of instruments and instruments being scattered and dropped off field during surgical procedures.

(g) A object of the present invention is to save valuable time lost when instruments are contaminated by falling off the field and needing resterelization.

(h) A object of the present invention is to provide a magnetic instrument bar which can be safely placed where needed.

These and other objects and advantages of the present invention, along with features of novelty appurtenant, will appear or become apparent in the course of the following descriptive sections.

### BRIEF DESCRIPTION OF DRAWINGS

In the following drawings, which form a part of the specifications and which are to be construed in conjunction therewith, and in which like reference numerals have been employed throughout wherever possible to indicate like parts in various views:

FIG. 1 is a side end sectional view of my Magnetic Instrument Bar;

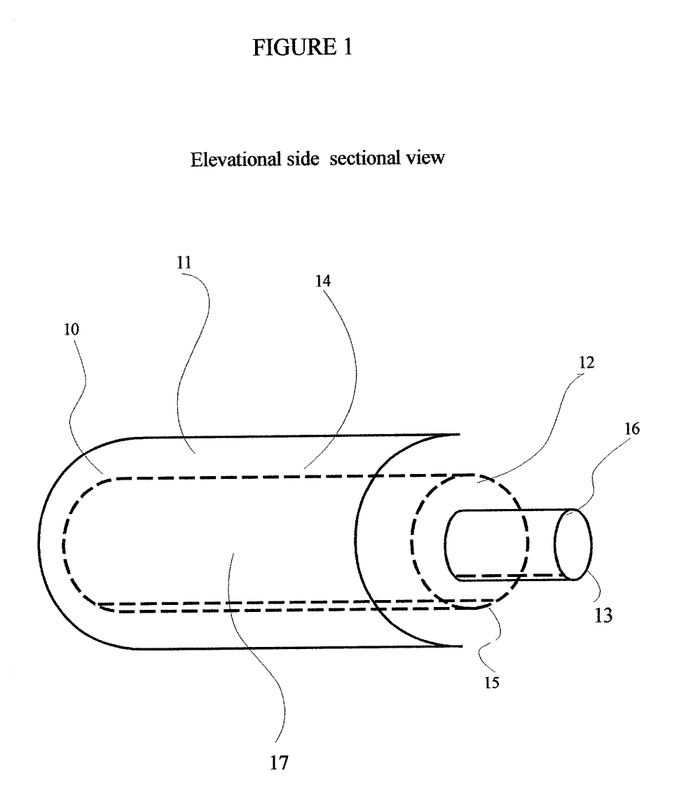

FIG. 2 is a top elvational view of my Magnetic Instrument Bar;

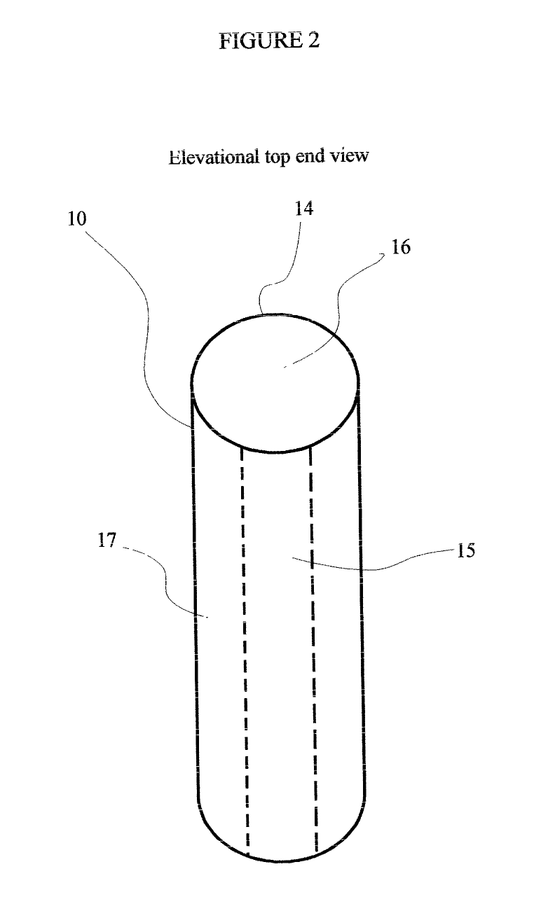

FIG. 3 is a bottom elevational view of my Magnetic Instrument Bar;

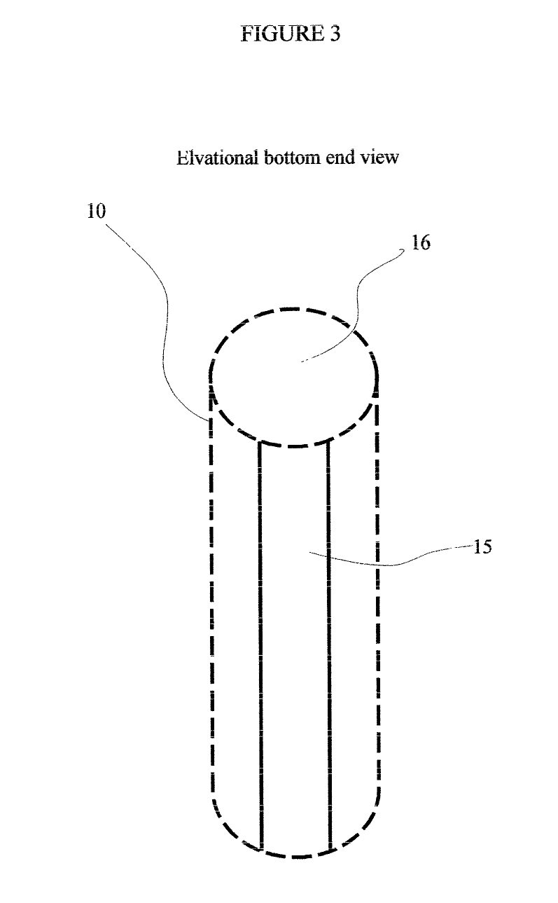

FIG. 4 is a side end elevational view of my Magnetic Instrument Bar;

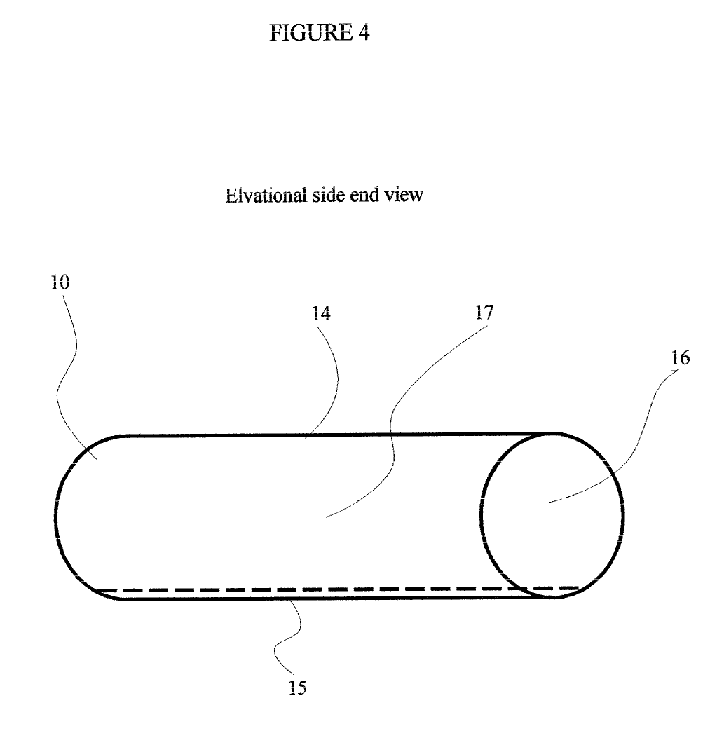

FIG. 5 is a end plan view of my Magnetic Instrument Bar;

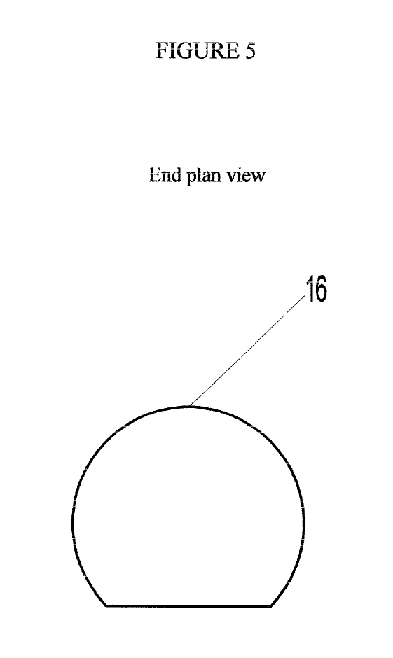

FIG. 6 is a top environmental view of my Magnetic Instrument Bar on a flat table surface with instruments in place;

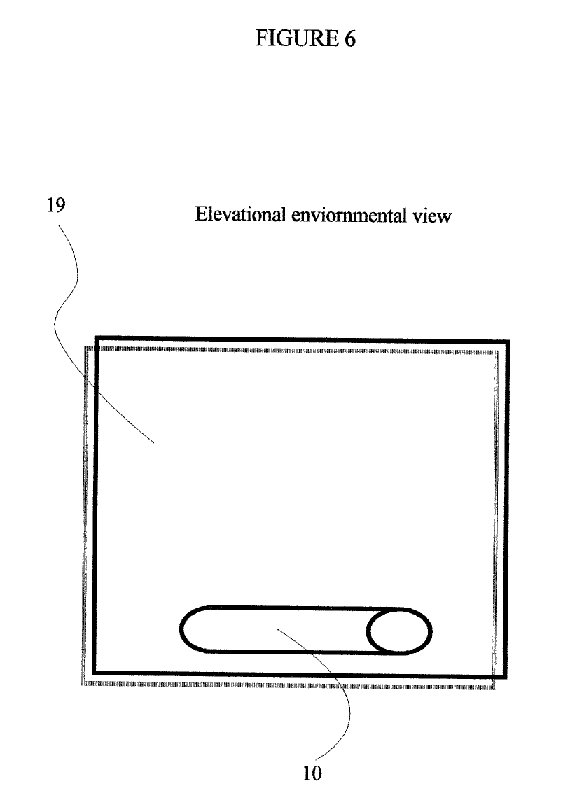

FIG. 7 is a environmental view of my Magnetic Instrument Bar deployed on a flat table surface with instruments in place;

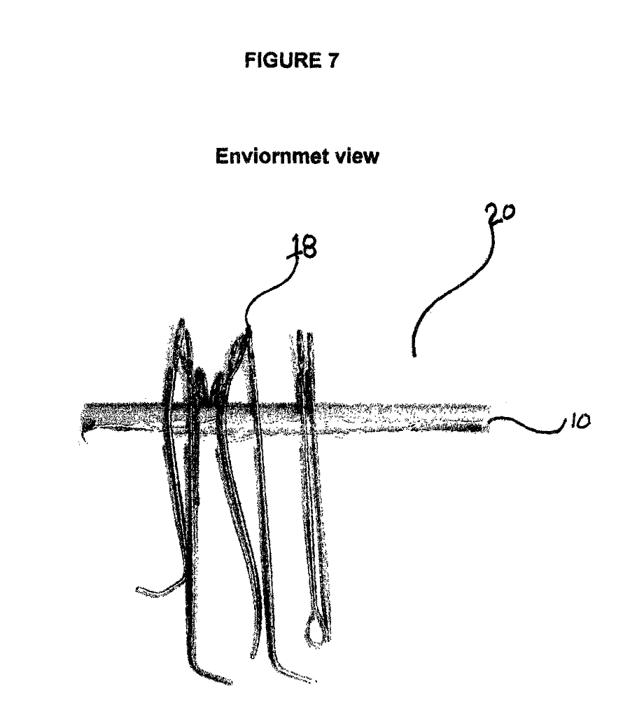

### REFERENCE NUMERALS IN DRAWINGS

10 elongated horizontal bar

11 rubber material

12 magnetic material

13 durable stainless steel

14 rounded top area of bar

15 flat bottom area of bar

16 smooth flat ends of bar

17 smooth rounded sides of bar

18 surgical instruments

19 flat table surface

20 instruments on bar on table

### DETAILED DESCRIPTION OF DRAWINGS

With reference now to the accompanying drawings, the preferred embodiment of my Magnetic Instrument Bar is broadly designated by the reference numeral 10 my Magnetic Instrument Bar, 10 generally comprises an elongated horizontal rounded bar with a 15 flat bottom having a 11 durable rubber material covering outer most section 12 a durable magnetic material section middle segment and a 13 a durable surgical stainless steel material bar inner most segment 14 the top rounded area of the magnetic instrument bar 15 the flat bottom section or base of the magnetic instrument bar 16 the flat smooth rounded end section of the magnetic instrument bar 17 the smooth rounded sides of the magnetic instrument bar 18 surgical instruments 19 a flat surface table 20 an environmental view of instrument on magnetic instrument bar.

Preferably, 13 the inner most segment is made of a durable surgical stainless steel or equivalent material suitable for repeated sterilization. 12 the middle segment is made of a magnetic material suitable for repeated sterilization and enough magnetic integrity to hold instruments in an upright position 11 outer most layer is made of a durable rubber material suitable for repeated sterilization and is a smooth surface that covers all surfaces.

The magnetic instrument bar simplifies successful upright positioning of surgical and non-surgical instruments for all procedures. The magnetic instrument bar is sterilized prior to each use.

To deploy the magnetic instrument bar the field or surface area is draped in the usual fashion and the magnetic instrument bar is then positioned in the desired area of the sterile field, instruments are then placed on top of the bar.

Thus, deployed the magnetic instrument bar operates as a instrument holder, wherein the magnetic force holds the instruments in an upright position.

From the foregoing, it will be seen that this invention is one well adapted to obtain all ends and objects herein set forth, together with other advantages which are inherent to the structure.

It will be understood that certain features and subcombinations are of utility and may be employed without reference to other features and subcombinations. This is conti plated by and is within the scope of the claims.

As many possible embodiments may be made of the invention with out departing from the scope thereof, it is to be understood that all matter herein set forth or shown in the accompanying drawings is to be interpreted as illustrative and not in a limiting sense. For example, the use of a variety of material to construct the present invention is anticipated.

What is claimed is:

1. A Magnetic Instrument Bar Comprising:
(a) an elongated body member with a generally smooth rounded top, a flat bottom and ends comprising of a generally horizontal bar consisting of a durable rubber material outer layer, a durable magnetic center layer and a durable surgical stainless steel material most inner layer.
(b) a magnetic instrument bar as defined in claim 1 wherein said outer layer comprises of a durable rubber material with the integrity to withstand repeated sterilization.
(c). a magnetic instrument bar as defined in claim 1 wherein said middle layer comprises of durable magnetic material with the integrity to withstand repeated sterilization.
(d) a magnetic instrument bar as defined in claim 1 wherein said inner most layer is comprised of a durable surgical stainless steel material with the integrity to withstand repeated sterilization.

2. The magnetic instrument bar as defined in claim 1 wherein all surfaces and ends are rounded and smooth wherein to ensure safety of personnel an equipment.

3. The magnetic instrument bar of claim 2, wherein:
(a) a magnetic instrument bar as defined in claim 2 wherein said bottom portion is flat with rounded smooth edges.
(b) a magnetic instrument bar as defined in claim 2 wherein said top portion is a smooth rounded surface.
(c). a magnetic instrument bar as defined in claim 2 wherein said end portions are a flat with a smooth surface.

4. The magnetic instrument bar of claim 3, wherein the said elongated body member is in the range of about 8 to 24 inches; and the circumference of the elongated body is in the range of about ¾ to 4 inches.

5. A method of handling a plurality of surgical instruments comprising:
(a) placing said instruments onto an instrument holder, said magnetic instrument holder including an elongated body member having an upraised cross section and magnetic interior portion. The magnetic force holds an instrument upright once placed against on said bar.

6. The method of claim 5, further comprising;
(a) viewing the instruments within said magnetic instrument bar, the bar being adapted to display the instruments, the instruments being disposed generally parallel to one another to facilitate viewing of said instruments.
(b) selecting a desired instrument for removal from the instrument holder, and
(c) removing the desired instrument from said magnetic instrument bar, the instrument bar being adapted to permit removal of instruments, one end of each instrument being disposed higher than the other end thereof facilitate grasping of said instrument.

7. The method of claim 6, wherein said magnetic instrument bar is sterilized and aseptically packaged prior to sue so that it can be stored in a conventional manner and then opened when needed to hold medical instruments.
